(19) United States
(12) Patent Application Publication
(10) Pub. No.: US 2007/0083126 A1
(43) Pub. Date: Apr. 12, 2007

(54) Apparatus & method for determining physiologic characteristics of body lumens

(75) Inventors: Alexei Marko, Vancouver (CA); Ian McDougall, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Angiometrx, Inc., Vancouver (CA)

(21) Appl. No.: 11/235,803

(22) Filed: Sep. 27, 2005

(51) Int. Cl. A61B 5/02; A61M 31/00

(52) U.S. Cl. 600/505; 604/509

(57) ABSTRACT

Disclosed are a system, apparatus, and method for determining a physiologic characteristic of a body lumen that include determining, at one or more selected pressures, the volume of incompressible medium infused into a balloon 54 of catheter 24 while the balloon is placed in each of (a) a lumen having a predetermined, fixed diameter and (b) a desired location of a body lumen having an unknown diameter. In certain variations, the physiologic characteristic (e.g., diameter, cross-sectional area) is determined by calculating the difference in the volume of infused medium between (a) and (b) at at least one static pressure. Other physiologic characteristics (e.g., compliance) are determined by calculating the difference in infused medium for (b) at at least two static pressures.

### BACKGROUND OF THE INVENTION

The present invention relates generally to the field of medical diagnostics. More particularly, the present invention relates to methods and devices for determining physiologic characteristics of body lumens, such as the diameter and wall compliance of blood vessels.

Many bodily diseases and/or abnormalities can be diagnosed by measuring the condition, i.e. size and/or compliance, of body members. One example is the vascular disease atherosclerosis, a progressive and degenerative process in which cholesterol and other fatty materials are deposited on the walls of arteries, forming a build-up of plaque known as a lesion. The accumulation of plaque narrows the interior passage, or lumen, of the blood vessels and in many cases impairs blood flow beyond the blockage. Atherosclerosis in the coronary arteries, which carry oxygenated blood to the heart, results in chest pain, known as angina, and can ultimately lead to heart attack and death. In peripheral arteries (the vascular system remote from the heart) atherosclerosis can lead to decreased mobility, chronic pain and amputation. In the cerebrovasculature (the vascular system of the brain) atherosclerosis can lead to stroke.

Atherosclerosis has usually been diagnosed using angiography techniques. Typical angiographic methods involve inserting a catheter into the vessel of interest and then injecting a contrast agent into the vessel through the catheter. The blood flow will carry the contrast agent along the vessel so that the vessel can be radiographically imaged with a display device such as a fluoroscope. The radiographic image of the vessel is then reviewed in order to estimate the internal diameter of the vessel to determine if there is any abnormal narrowing of the vessel which may have occurred due to disease. If any narrowing is observed, the extent (percentage) of narrowing is estimated from the radiographic image by measuring the vessel diameter both at and immediately before the region of narrowing with a ruler, calipers or similar device. Such a measurement is typically not particularly accurate since it relies on discerning an ill-defined boundary in a single plane. Additionally, stenotic material outside of the image plane can be missed. These limitations with the radiographic image typically result in average errors of approximately 30%. Such inaccuracy hinders adequate characterization of vascular disease.

Depending on the number, severity and location of the atherosclerotic obstructions, the physician may pursue a number of treatment options, including catheter-based interventional techniques. The majority of these catheter-based techniques involve a balloon tipped catheter being inserted through the artery and into the obstruction. Once the balloon is centered within the narrowed section of artery, it is inflated, pushing the plaque into the artery wall and opening or “dilating” the vessel in order to restore normal blood flow. The balloon is usually inflated several times in order to completely open the artery.

In the majority of angioplasties performed today, the balloon is used in conjunction with a “stent”—a tiny metal structure which is expanded and left in place within the artery to hold the artery open after it has been dilated.

In order to perform these procedures it is advantageous for the physician to have a precise measurement of the size of the vessels at the area being treated. This information allows them to best quantify the extent of a blockage within the vessel, select appropriate angioplasty balloon and stent sizes for treatment, and confirm adequate dilation of the plaque following the intervention.

Radiographic assessment of, e.g., stent expansion is subject to the above-described and additional errors that can further hinder the physician's ability to deliver adequate treatment to the patient. When this technique is used to determine the proper expansion of a stent implanted in the artery, measurements are complicated by the fact that, if the stent is under-expanded, the contrast agent can flow freely through the mesh of the stent wall, which can give the appearance of full expansion, even though the stent is not fully apposed against the artery wall.

Another significant vascular condition known as “hardening of the arteries” typically occurs with aging and is characterized by the vessel wall becoming rigid, resulting in a lost capacity to expand and contract during the cardiac cycle. Normally, the vessel wall is sufficiently compliant that it expands as blood pressure rises and contracts as blood pressure falls within each cardiac cycle. It would be quite useful to accurately measure the compliance of vessel walls to determine the location and extent of non-compliant portions of the vascular system. It would be particularly useful to make such a determination prior to any interventional therapy, such as balloon angioplasty or stent implantation, that results in physical alteration of the atheroma and/or blood vessel wall. Such early determinations would be of great value in selecting the mode of interventional therapy, best suited for the patient's condition.

For example, several recent clinical studies have suggested that proper vascular stent deployment directly affects clinical outcome, and the rate of re-stenosis. (See Fizgerald et al., Circulation 102:523-530, 2000; Russo et al., Circulation 100:I-234, 1213, 1999; De Jaegere et al., European Heart Journal 19:1214-1223, 1998.) These studies suggest that angiography alone is not sufficient to ensure proper vascular stent deployment, and that re-stenosis rates will decline if proper stent apposition has occurred. The importance of proper stent sizing and apposition is further emphasised with the increasing use of drug eluting stents, the drug effects of which are only realized upon contact with the arterial wall.

Recent research also indicates that certain kinds of “vulnerable plaque” may trigger acute coronary events, creating a demand for new diagnostic tools and therapeutics. At least some coronary disease is now believed to be an inflammatory process, in which inflammation causes plaque to rupture. These so called “vulnerable plaques” don't at first block the arteries. Rather, much like an abscess, they are ingrained under the arterial wall, so that they are undetectable—they can't be seen by conventional angiography or fluoroscopy and they don't cause symptoms such as shortness of breath or pain. Yet, for a variety of reasons, they are more likely to erode or rupture, creating a raw tissue surface that forms scabs. Thus, they are more dangerous than other plaques that cause pain, and may be responsible for as much as 60-80% of all heart attacks. It would be ideal if the physician could “palpate” or feel the vessel from the inside out in order to detect the presence of the plaques or “soft spots” in the arterial wall.

The accurate measurement of both size and compliance of other body members, including the intestines, the bronchia, the urethra, and the cervix, among others, would assist in the determination of particular conditions and/or the diagnosis of disease in those members as well. Such size and compliance measurements would also assist in the determination of the proper size and in the deployment of therapeutic devices to be implanted in the target lumens.

Hence, there is a significant need for a system capable of accurately and directly determining in vivo the size and optionally the compliance of blood vessels, as well as other body members. Such a system should be relatively simple to use and relatively inexpensive, to accommodate a single-use strategy. The present invention as described hereinbelow meets these and other needs.

One proposed system for determining size and compliance of blood vessels is described in U.S. Pat. No. 5,275,169 to Afromowitz et al., which provides methods and devices for determining physiologic characteristics of body lumens. While the system described in U.S. Pat. No. 5,275,169 has been successful, further improvements are still needed.

### SUMMARY OF THE INVENTION

The present invention provides a method for determining a physiological characteristic at a location within a body lumen. The method generally includes the following steps: (1) introducing an incompressible medium into a balloon to inflate the balloon to at least one preselected pressure while the balloon is within a calibration lumen having a predetermined, fixed diameter; (2) determining the internal volume of the balloon while inflated within the calibration lumen at the preselected inflation pressure; (3) introducing the balloon while uninflated to the location within the body lumen; (4) introducing the incompressible medium into the balloon to inflate the balloon to the preselected pressure at the location; (5) measuring the internal volume of the balloon while inflated at the location; and (6) determining the physiological characteristic at the location based on the difference between the volume of the balloon while inflated within the calibration lumen and the measured volume at the location. In a preferred embodiment, the step of introducing incompressible medium includes introducing the incompressible medium by positive displacement and the volume measuring step includes measuring the volume based on the amount of incompressible medium introduced. In various embodiments, the body lumen is, e.g., a blood vessel, the gastro-intestinal tract, the bronchia, the urethra, or the cervix.

In certain embodiments of the method, the internal volume of the balloon while inflated within the calibration lumen is substantially zero. Calibration lumens that are suitable for achieving a substantially zero internal volume include, for example, a protective sheath or crimped stent, which can be assembled on the balloon during manufacturing of the balloon.

In some embodiments, the preselected pressure is below 300 mmHg. For example, where the body lumen is a coronary blood vessel, the preselected pressure is typically between 200 and 300 mm Hg. In other variations, where the body lumen is a non-vascular body lumen, the preselected pressure is typically less than one atmosphere. The method can also include determining medium volume for a plurality of medium pressures (e.g., a plurality of pressures below 300 mmHg).

Physiological characteristics of a body lumen that are particularly amenable to measurement using the method described herein include, for example, an internal dimension (such as, e.g., a cross-sectional area or an internal diameter) and compliance. Lumen compliance is typically determined based on a difference between a first measured volume of inflation medium at a first preselected pressure and a second measured volume at a second preselected pressure. In certain embodiments, the first and second preselected pressures are below 300 mm Hg. Determining lumen compliance can include calculating cross-sectional area and/or internal diameter at the first and second preselected pressures. In a specific variation, the second preselected pressure is greater than the first preselected pressure, and determining lumen compliance includes calculating a percentage increase between the first measured volume and the second measured volume.

The step of introducing incompressible medium can include, for example, introducing the medium at a substantially constant rate or, alternatively, at a substantially variable rate. In one specific embodiment, the substantially constant or substantially variable rate is in the range from 4 to 100 μl/sec.

The method for determining a physiological characteristic of a body lumen can also include the steps of stopping and then reversing the introduction of incompressible medium into the balloon at preselected values of medium pressure and volume. In particular variations of this embodiment, the preselected medium pressure is in the range from 0 to 400 mmHg. The incompressible medium can be, for example, introduced by positive displacement and the volume measured based on the amount displaced.

The present invention also provides a system for measuring a physiological characteristic of a body lumen. The system generally includes (1) a catheter body having a proximal end, a distal end, and an inflation lumen extending from the proximal end to near the distal end; (2) an inflatable balloon mounted near the distal end of the catheter body and being connected to the inflation lumen; (3) means for introducing a volume of an incompressible medium through a proximal end of the inflation lumen to inflate the inflatable balloon; (4) means for measuring the pressure of incompressible medium within the balloon; and (5) means for determining the physiological characteristic of a location within the body lumen based on the difference between (a) the volume of inflation medium required to produce a preselected pressure of inflation medium within the balloon when the balloon is constrained within a lumen having a predetermined, fixed diameter and (b) the volume of inflation medium required to produce the preselected pressure of inflation medium within the balloon when the balloon is at the location within the body lumen. In one preferred embodiment of the system, the means for introducing a measured volume of inflation medium includes a syringe. The syringe can be coupled to, e.g., a linear actuator.

In certain embodiments, the means for measuring pressure includes a pressure sensor disposed within the interior of the inflatable balloon. In an alternative embodiment, the means for measuring pressure includes a pressure sensor disposed at or near the proximal end of the inflation lumen. In some variations of the system, the means for measuring the pressure of incompressible medium within the balloon typically includes means for measuring a pressure of up to about 400 mm Hg.

A particularly suitable catheter body includes at least a second lumen that is connected at its distal end to the interior of the inflatable balloon. In these embodiments, the means for measuring pressure is typically connected to the second lumen. For example, the second lumen can extend to the proximal end of the catheter, and the pressure measuring means can include a pressure sensor disposed near the proximal end of the second lumen.

In typical embodiments of a system configured for measuring coronary blood vessels, the inflatable balloon when inflated is generally cylindrical and has a length in the range from about 3 mm to about 15 mm and a diameter in the range from about 2 mm to about 10 mm.

In yet another aspect, the present invention provides a system, for determining a physiologic characteristic of a body lumen, for connection to a balloon catheter, the balloon catheter having a proximal end, a distal end, an inflation lumen extending from the proximal end to near the distal end, and an inflatable balloon mounted near the distal end of the catheter body and connected to the inflation lumen. The system generally includes (1) means for introducing a measured volume of incompressible medium to the balloon catheter; (2) means for receiving a signal corresponding to static pressure within the balloon from the catheter; and (3) means for calculating the physiologic characteristic based on (a) the value of a first measured volume of inflation medium at a preselected pressure when the inflatable balloon is constrained within a lumen having a predetermined, fixed diameter, and (b) the value of a second measured volume of inflation medium at the preselected pressure when the inflatable balloon is disposed within the body lumen. In certain embodiments, the means for introducing includes a syringe operatively connected to a position controlled motor. Further, the means for calculating can include, for example, a microprocessor that is connected to both the positioned controlled motor and the pressure means for receiving.

### BRIEF DESCRIPTION OF THE DRAWINGS

FIG. 1 is a block diagram showing a the system of the present invention.

FIG. 2 shows a console and balloon catheter of the system of the present invention.

FIGS. 3A-3C show an infusion syringe and syringe clamps of one exemplary infusion system of the present invention. FIG. 3A depicts the syringe clamps open. FIG. 3B depicts the infusion syringe with syringe adaptor. FIG. 3C depicts the syringe clamps closed with the syringe in place.

FIG. 4 shows one exemplary balloon catheter of the present invention.

FIG. 5 shows one exemplary balloon of the catheter of the present invention.

FIG. 6 shows one embodiment of the balloon catheter shaft-section immediately proximal to the balloon.

FIG. 7 is a simplified flow diagram showing one method for measuring a physiologic characteristic of a body lumen.

FIG. 8 shows one exemplary block having a plurality of fixed lumens for calibration of the balloon catheter according to the system and methods of the present invention.

FIG. 9 is a set of ideal curves of fluid volume infusion against pressure for the system of the present invention, when the balloon portion of the catheter is unrestrained and then inside fixed lumens of various sizes.

FIG. 10 is a set of actual recorded curves of fluid volume infusion against pressure for the system of the present invention, when the balloon portion of the catheter is inside various sizes of rigid tubing.

FIG. 11 is a set of actual recorded curves of fluid volume infusion against pressure for the system of the present invention, when the balloon portion of the catheter is inside a fixed lumen of known size and a blood vessel of a patient having a blood pressure of around 135/65 mmHg.

FIG. 12 graphically depicts a size and a compliance determination according to the methods of the present invention. Curves of fluid volume infusion against pressure for the system of the present invention are shown when the balloon portion of the catheter is inside in a fixed lumen of known diameter (the calibration curve) and when the balloon portion of the catheter is inside a body lumen being measured (the measurement curve).

### DETAILED DESCRIPTION OF THE INVENTION

For the purposes of the present specification and claims, the following terms and phrases are defined as follows.

The phrase “body lumen,” as used herein, includes all hollow body organs, vessels, passages, and the like, particularly including blood vessels, the gastro-intestinal tract, the bronchia, the urethra, the cervix, and the like.

The phrase “static pressure,” as used herein, refers to the gauge pressure, i.e., pressure above ambient, within the balloon which is free from pressure transients, back pressure (dynamic pressure drop), and the like, at a specific instance in time. Static pressure within the balloon is thus preferably measured by a pressure sensor element within the balloon itself or by a pressure sensor located within a separate pressure measurement lumen where a static column of fluid can be maintained between the balloon and the pressure sensing element. It will also be possible to measure the static pressure at the inlet of the balloon inflation lumen or at other locations within the catheter (as described in more detail below), but any transient pressure variations, such as pressure drop during fluid infusion or the ambient pressure relative to atmosphere that is acting on the balloon at the measurement site (such as pressure due to patient blood pressure), must be taken into account in determining the true internal pressure of the balloon.

The phrase “measured volume,” as used herein, refers to the volume of incompressible inflation medium pumped into the catheter lumen for inflation of the balloon in accordance with the methods of the present invention. Accordingly, any reference herein to “measured volume” within the balloon is understood to include the volume of inflation medium within the balloon itself as well as the volume within the catheter shaft extending from the balloon to the infusion pump. Usually, the volume will be measured using a calibrated positive displacement mechanism for introducing the incompressible inflation medium to the balloon, usually a calibrated syringe which may be driven by a stepper motor or a servo-controlled motor. In the case of internal dimension measurements, it will be necessary to determine the absolute volume of fluid within the balloon, excluding volumes within the inflation lumen, pressure measurement lumen (if employed), fluid introducing means, and the like. In the case of wall compliance measurements, it will usually be necessary to measure only differential volume between two or more different values of static pressure.

The phrase “incompressible medium,” as used herein, refers to any incompressible substance that can flow and conform to the shape of a container. The phrase “incompressible fluid,” as used herein, refers to an incompressible medium that is a fluid, typically a liquid, and includes, for example, a variety of liquids of the type normally employed for the inflation of intravascular balloons (e.g., angioplasty balloons). Exemplary incompressible fluids include, e.g., sterile water, saline, contrast media (typically diluted with water), and the like. Typically, there is some volume of air bubbles present in this medium, which allows some small degree of compressibility; however, this degree of compressibility is sufficiently small that it can be ignored.

The present invention comprises methods, systems and apparatus for determining a physiologic characteristic, such as an internal dimension or wall compliance, of a body lumen, such as a blood vessel, the gastro-intestinal tract, the bronchia, the urethra, the cervix, or the like. The method relies on introducing an incompressible medium to a balloon and monitoring the static pressure and total volume of incompressible medium within the balloon. The volume of incompressible medium is measured at a pressure when the balloon is constrained within a lumen having a predetermined, fixed diameter (also referred to herein as the “calibration volume”) and when the balloon is disposed at a location within a body lumen. The physiologic characteristic is then calculated based on the difference between the calibration volume and the measured volume at the location of the body lumen.

Advantageously, the methods are carried out at balloon inflation pressures that are at or only slightly above the average physiologic pressure for the particular body lumen. For arteries, the balloon is typically inflated to a pressure in the range from about 200 mmHg to about 300 mmHg, and preferably between about 250 mmHg and about 260 mmHg, with the particular value depending at least in part on vessel location and type. It is advantageous to use a pressure of greater than 200 mmHg when making measurements in arteries, since it permits the balloon to fully expand in order to conform to the shape of the vessel wall. Furthermore, since ambient pressure is typically less than 200 mmHg at the time of measurement, these pressures allow the balloon to overcome the fluid pressure within the lumen such that these pressures do not significantly act on the balloon to prevent it from fully expanding to fill and occlude the vessel over the length of the balloon. A pressure of about 250 mmHg is preferred for arterial measurements since it is above the peak blood pressure that is expected in the artery at the time of measurement, but is within the maximum range that might be expected in the patient during activities such as physical exertion and can therefore be considered within the “physiological range.”

In addition to vascular body lumens, the methods described herein can also be used to measure non-vascular body lumens, including, for example, the gastro-intestinal tract, the bronchia, the urethra, the cervix, or the like. As with blood vessels, the measurement pressure for a particular non-vascular body lumen will typically vary according the type of body lumen being measured, the measurement location, and/or the particular application involved. Generally, the measurement pressure is within or slightly above a pressure considered with the “physiological range” for the non-vascular body lumen, and is typically less than one atmosphere.

By employing relatively low pressures that approximate physiologic pressure, the methods of the present invention have a minimum impact on the characteristic being determined as well as on the physical structure of the lumen itself. In particular, with blood vessels, the structure of the plaque and blood vessel wall can be assessed without significant mechanical disruption (as would be the case with methods that determine wall compliance during high pressure angioplasty procedures). An optimum treatment strategy can then be selected.

Measurement of an internal dimension of the body lumen (e.g., cross-sectional area or diameter) is performed by measuring the total volume of an incompressible medium within the balloon at at least one static pressure, preferably within the range set forth above. The internal dimension is then calculated by comprising this volume of medium required to fill the balloon, where the static pressure is sufficient to cause the balloon to contact the interior wall of the lumen, to a volume of medium required to fill the balloon to the same static pressure in a lumen of know size. In the case of a generally cylindrical balloon, the average cross-sectional area of the lumen across the length of the balloon is calculated by dividing the volume by the known balloon length.

Measurement of body lumen wall compliance is performed by determining the total volume of incompressible medium within the balloon at at least two static pressures within the range set forth above. The wall compliance is then calculated based on the observed difference in volume of incompressible medium at the two static pressures.

Systems according the present invention generally comprise a catheter body having an inflatable balloon at or near its distal end. A device for introducing a measured volume of an incompressible inflation medium is connected to the balloon through an inflation lumen which extends from the proximal end of the catheter body. A device for measuring the pressure of the inflation medium within the balloon is provided, and another device is connected to both the introducing means and the measuring means for calculating the desired physiologic characteristic based on the measured volume of the inflation medium at one or more pressures approximating the described pressure range. A pressure measuring device comprising a pressure sensor is preferably disposed within a separate pressure measurement lumen within the catheter body. The preferred inflatable balloon is generally cylindrical and has a length which depends on the characteristic being measured, usually being in the range from about 3 mm to about 10 mm for dimensional measurements in blood vessels. The diameter will be slightly greater than that of the lumen being measured, typically being in the range from about 1 mm to about 8 mm for blood vessels.

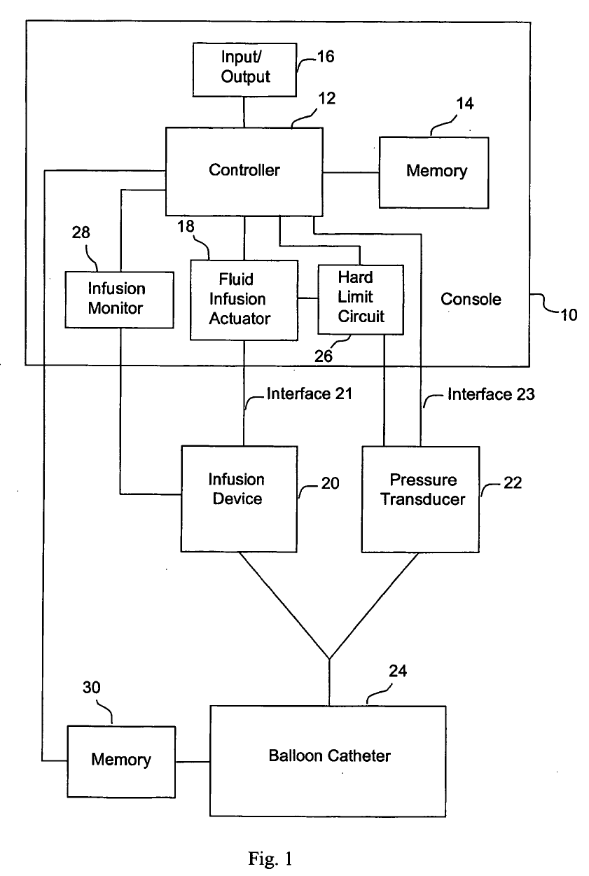

FIG. 1 schematically illustrates a preferred embodiment of the system in accordance with the present invention. The system includes a console 10 that comprises a controller 12 in communication with a memory 14, an input/output assembly 16, a fluid infusion actuator 18, and a pressure measuring device that is typically in the form of a pressure transducer 22 (via an interface 23). Fluid infusion actuator 18 is coupled via an interface 21 to an infusion device 20. A balloon catheter 24 is in fluid communication with each of pressure transducer 22 and infusion device 20. Fluid infusion actuator 18 drives the infusion device 20 to infuse fluid into balloon catheter 24. Optionally, the system further includes an infusion monitor 28, which is in communication with controller 12 and directly monitors infusion device 20 to detect the volume of infused fluid.

Input/output assembly 16 can include any conventional type of input and/or output, such as buttons, switches, knobs and/or the like, for operation of the system by the user, as well as, e.g., a display system (e.g., LCD display) for displaying information regarding any of various aspects of system operation.

In some variations, the system of FIG. 1 may include a hard limit circuit 26, in communication with each of controller 12, fluid infusion actuator 18, and pressure transducer 22. Hard limit circuit 26 may be configured to cut power to the fluid infusion actuator 18 when a predetermined pressure is measured by the pressure transducer.

The balloon catheter 24 may optionally include a memory 30, which will be in communication with controller 12 when the balloon catheter 24 is coupled to console 10. Memory 30 typically contains address spaces allocated for information specific to the balloon catheter. Memory 30 can be located anywhere on or within the balloon catheter 24, but is preferably integral to balloon catheter 24. As will be described below, in certain embodiments, memory 30 is contained within a pressure transducer connector 36 (see FIG. 4) that connects the pressure transducer to console 10.

The infusion of fluid into the balloon catheter is accomplished using fluid infusion actuator 18 that drives infusion device 20. Infusion device 20 is typically a positive displacement device such as a calibrated infusion syringe. The infusion device 20 may be driven by infusion actuator 18 in the form of a linear stepper motor that drives displacement (e.g., in the case of a calibrated syringe, displacement of the syringe plunger) with no need to provide feedback control. In alternative embodiments, infusion actuator 18 is integrated into a servo system (not shown) that is responsive to a predetermined fluid infusion rate schedule and a measured (feedback) value of actual infused fluid volume. The rate of infusion may be varied during the infusion process according to a predetermined pattern or schedule. The schedule may vary from console to console due to the inertial forces associated with starting and stopping the fluid infusion device 20. The use of rate schedules permits the cancellation of viscous flow effects associated with fluid flow in the catheter. In certain embodiments, the infusion rate is determined based on (e.g., as a function of) data acquired from memory 30.

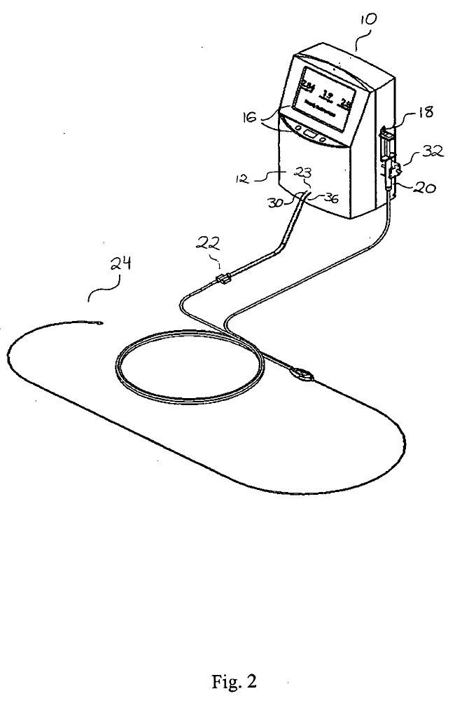

A system of the present invention comprising an infusion syringe is shown in FIG. 2, which shows console 10 (having input/output assembly 16), infusion syringe 20, pressure transducer 22, memory 30, balloon catheter 24, and a syringe connection assembly 32 for connecting the syringe 20 to infusion actuator 18. In certain embodiments, syringe connection assembly 32 is configured such that it will accept only the use of a calibrated syringe and will not accept connection of other syringes. FIG. 2 also shows a pressure transducer connector 36, which is in communication with pressure transducer 22 and couples pressure transducer 22 to controller 12 at interface 23. In certain embodiments in which pressure transducer 22 produces an electrical output, pressure transducer connector 36 is in electrical communication with pressure transducer 22.

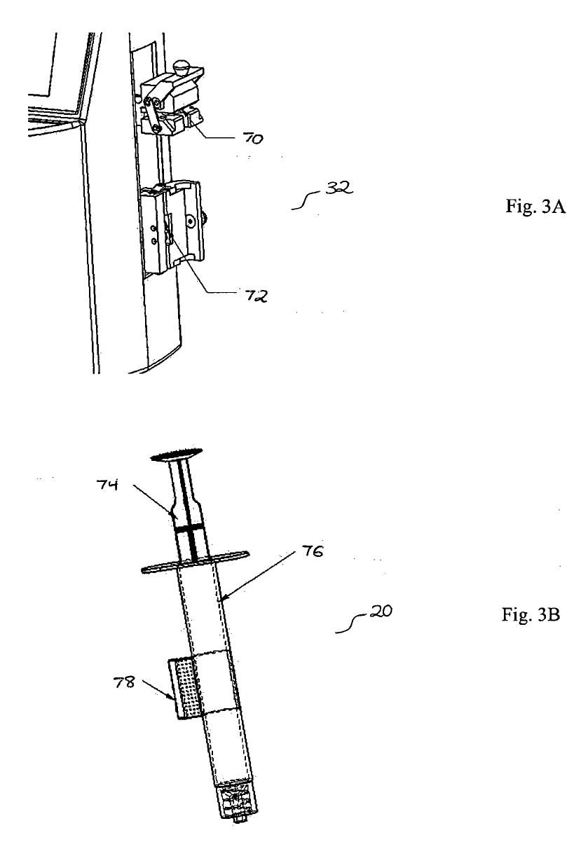

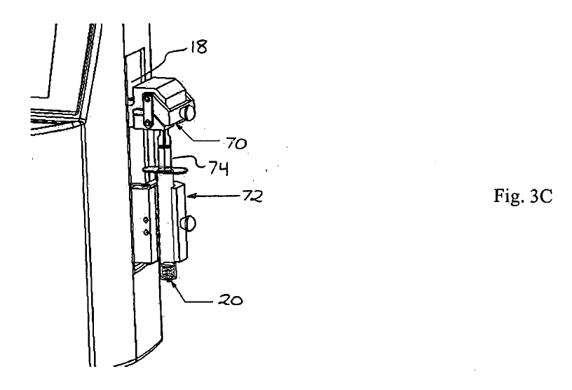

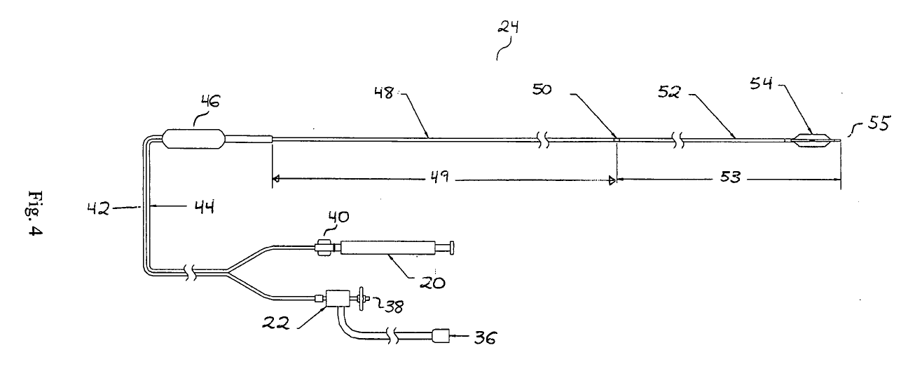

FIGS. 3A-3C show one embodiment of a syringe connection assembly. The syringe connection assembly 32 comprises a plunger clamp 70 and a barrel clamp 72 (FIG. 3A) for accepting, respectively, a syringe plunger 74 and a syringe barrel 76 of infusion syringe 20 (FIG. 3B). Syringe adaptor 78 is configured to mate with barrel clamp 72. Plunger clamp 70 and/or barrel clamp 72 can further comprise one or more detect switches (not shown) for detecting connection of the syringe. For example, in a specific variation, each of the plunger clamp and barrel clamp contain 2 detect switches. The detect switches form a closed circuit when the syringe 20 is properly placed in the syringe connection assembly 52 and both the plunger clamp 70 and barrel clamp 72 are closed. FIG. 3C shows the plunger clamp 70 and barrel clamp 72 closed with the syringe 20 in place. With the syringe in place as depicted in FIG. 3C, infusion actuator 18 in the form of a stepper motor drives displacement of the plunger clamp 70 and the syringe plunger 74, thereby infusing fluid into the balloon catheter 24 and eventually into a balloon 54 (FIG. 4).

In one specific embodiment, 10-200 microliters (μl) of fluid is infused at a preselected rate of between about 4 to about 12 μl/sec and the pressure is monitored by the pressure transducer within the balloon catheter 24 over the range of about −100 to about 350 mmHg. The fluid in the syringe should be sterile, relatively gas-free, and substantially incompressible. The action of syringe 20 may optionally be detected by a displacement monitor 28 which confirms the actual infused volume. For example, in certain embodiments in which infusion pump 18 is in the form of a linear stepper motor drive coupled to plunger clamp 70, displacement monitor 28 monitors displacement of the stepper motor drive. At start-up, fluid is drawn back until a pressure of −100 mmHg is reached, and this position is then set as the “zero” volume position. Thereafter, infused volume is determined with reference to this zero volume position, as the linear displacement of the plunger clamp 70 (determined, e.g., by counting steps) multiplied by the cross-sectional area of the syringe barrel (typically a parameter contained on memory 30 and accessed by controller 12), thereby yielding the infused volume.

It should be understood that both the fluid infusion rate schedule and the total infused volume can be varied depending upon the application. For example, in certain embodiments, larger balloons and fluid volumes are used to measure larger lumens. Also, it should be understood that other arrangements and/or other components could be used for fluid infusion.

FIG. 4 shows one specific embodiment of balloon catheter 24 in more detail. Balloon 54 is in fluid communication with a distal shaft 52, a proximal shaft 48, and extension lines 42 and 44. An adapter 46 at the proximal end of the catheter transitions the proximal shaft 48 to extension lines 42 and 44. Extension line 42 connects to the pressure measurement lumen, which extends from the balloon and is in communication with pressure transducer 22. Extension line 44 connects to the balloon inflation lumen that receives the infused fluid from syringe 20. The balloon catheter further includes guidewire entry port 50 located proximal to the distal tip of the catheter. The distal tip of the catheter also includes a guidewire exit port 55. The working length of the balloon catheter comprises proximal shaft 48, distal shaft 52, and balloon 54 (the distance between the adaptor 46 and the distal guidewire exit port 55). To facilitate positioning of the catheter, section 53 (defining a section of the catheter between guidewire entry port 50 and guidewire exit port 55) is typically more flexible than section 49 (defining a section between adaptor 46 and guidewire entry port 50). Typically, the balloon catheter has a working length between about 60 cm and about 200 cm. In one specific embodiment, the balloon catheter has a working length of about 140 cm, with distal section 53 having a length of about 30 cm and proximal section 49 having a length of about 110 cm.

The proximal end of extension line 44 has a fitting 40 for attachment to the infusion syringe 20. The proximal end of extension line 42 is coupled to pressure transducer 22, which has a fluid shut off valve 38 at its proximal end. Pressure transducer 22 is in communication with pressure transducer connector 36, which couples pressure transducer 22 to controller 12 at interface 23 as outlined in FIG. 1.

Pressure transducers particularly suitable for use in the present invention are standard pressure transducers typical of those used for invasive blood pressure monitoring in patients. The pressure transducer 22 is typically disposable and an integral part of the disposable catheter. Alternatively, a microminiature pressure transducer could be positioned in the opening of pressure lumen 68 (see FIG. 6) near balloon 54 (see FIG. 4) or within balloon 54 itself in a particular application. Semiconductor strain gauge pressure transducers or fiber optic pressure sensors are available in sufficiently small sizes for such alternative embodiments.

The patient-contacting parts of the catheter are preferably compatible with short-term, invasive, externally communicating, direct contact with the body tissue (e.g., blood, vascular tissue). The indirect contacting parts are preferably selected for short-term, indirect contact of the tissue, and non-patient contacting parts are preferably selected for short-term skin contact. Particularly suitable materials for use in the balloon catheter include, e.g., nylon, stainless steel, polyimide, polyvinyl chloride (PVC), and polycarbonate, plus cyanoacrylate adhesives. The pressure transducer typically does not have direct or indirect contact within the body tissue. In a preferred embodiment, there is a silicone membrane between the balloon inflation fluid medium and the pressure transducer. Preferably, the balloon catheter further includes a stiffening wire (not shown) that extends from adapter 46 to the guidewire entry port 50. The stiffening wire helps give the proximal section 49 of the design stiffness for push-ability and reduces the likelihood of kinking the catheter.

The balloon 54 can be similar to that which is conventionally used for the intravascular angioplasty technique, provided that the balloon is shortened and the material and wall thickness are selected to facilitate balloon operation at low fluid pressures. Conventional balloons used for angioplasty are relatively long while the balloon of the present invention will vary between a few millimeters to several centimeters (typically having a length from about 3 to about 15 mm for dimensional measurements in blood vessels) and hence can be more location specific along the vessel being measured. The balloon diameter will be slightly greater than the largest lumen being measured (typically being from about 2 mm to about 4 mm for dimensional and/or compliance measurements in coronary blood vessels and in the range of about 3 to about 12 mm for peripheral vessels such as those in the limbs of a human subject). In its fully inflated state, the balloon will form a cylinder, the length of which will typically be in the range of 3-15 mm.

Balloon 54 of the catheter typically withstands a pressure of up to at least 760 mm Hg, more typically up to at least 350 mm Hg, without significant stretching, yet is also flexible enough to conform to irregularities in the arterial walls, and unfold and open upon fluid infusion without significant resistance. Conventional angioplasty balloons are designed to withstand 6-15 atmospheres of pressure (one atmosphere equaling 760 mmHg). The angioplasty balloon material is necessarily fairly thick (typically approximately 3 mils) to prevent rupture. However, a thick wall balloon does not readily conform to vessel internal geometry. Additionally, thick wall balloons are characterized by irregular balloon fluid pressure during inflation, due to the unfolding of the stiff balloon material. A low pressure balloon may be made of thinner material (1 mil or less), resulting in the balloon having improved conformance to vessel walls and reducing balloon unfolding pressure artifacts during inflation. Thinner material allows the balloon to be folded in such a manner to further reduce inflation artifacts. A low pressure balloon thus will likely provide more accurate, reliable measurements near or in the physiologic pressure range, including measurements within lumens having non-uniform (e.g. non-cylindrical) cross-sections.

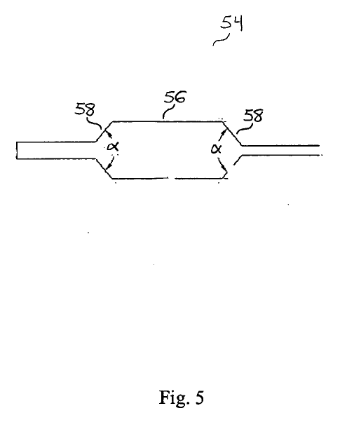

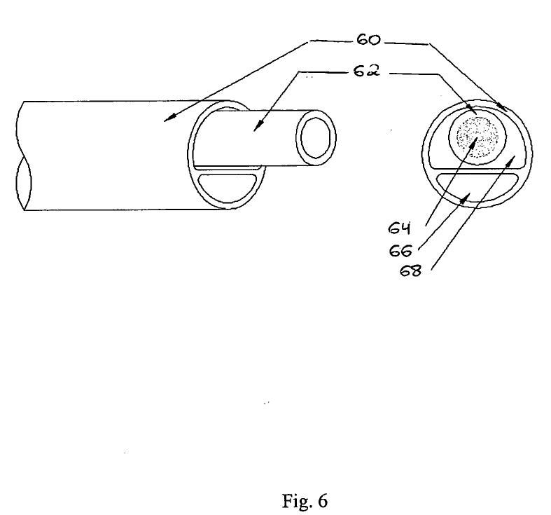

Balloon 54 is essentially non-compliant (i.e., does not significantly deform or stretch) at the pressure range used by the system of the present invention. Typically, balloon 54 is generally cylindrical in shape and manufactured from a polymer material that allows it to have a small, uninflated profile yet assume a known diameter at low inflation pressures. In a preferred embodiment, radio-opaque markers are positioned under the balloon for fluoroscopic identification and positioning. The markers demark the section of the balloon that contacts the body lumen (e.g., artery) during the measurement cycle; cross-sectional area and diameter are then averaged over this section of balloon 54 that makes contact with the vessel wall. FIG. 5 shows a specific embodiment of balloon 54 in more detail, having a generally cylindrical shape at the midsection 56 with tapered ends 58. Each tapered end 58 of the balloon defines angle α. In one particular embodiment, angle α is 100°.

The balloon 54 of the present invention is designed to operate at much lower pressures compared to typical angioplasty balloons, typically within the range of 0-350 mm Hg for arterial measurement applications, since this is near the range of pressures normally present in a these blood vessels. For measurements in other body lumens such as the urethra or bronchia it is expected that the ideal pressure will vary but will typically be in a range of less than 1 atmosphere. Typically, balloon 54 is designed to operate at a pressure slightly above the physiologic pressure range normally experienced by the artery, but within the extremes of the range which the artery might be expected to experience overall or slightly above this range. The typical range of peak human arterial blood pressure is 100-180 mm Hg, with peak systolic blood pressures over 250 mm Hg observed during stress testing or physical exertion. Inflating the balloon to a pressure higher than the actual physiologic pressure of fluid (blood) within the artery is desirable so as to ensure that the balloon comes into contact with, and is thereby constrained by, the lumen wall. Since the measurement of artery condition, such as cross-sectional area, is averaged over the entire length of the balloon, a shorter balloon length will allow measurement of cross-sectional area over a short distance, approximating a point measurement. This facilitates accurate mapping of disease along the length of the artery, particularly since such disease may be unevenly distributed along the artery. Similarly, this facilitates assessment of the expansion of a stent implanted in the artery, since it is frequently found that small sections of a stent which may only be a few millimeters in length are underdeployed where they meet significant resistance during dilation. Further, the balloon can be made in several different sizes (outside diameter) so that measurements of small, medium, and relatively large arteries (or veins or other tubular body members) can be more readily accommodated. Normal requirements of stiffness/flexibility apply to the catheter body itself for intravascular maneuvering (e.g., tracking over a typical guidewire).

As is typical of intra-arterial balloon catheters, the balloon is typically folded in such a manner that, when deflated, it maintains a minimal cross-section and can be readily navigated through small arteries. Upon expansion, these folds disappear as the balloon fills with fluid until it conforms to the shape of the lumen being measured.

FIG. 6 shows a balloon catheter shaft-section proximal to the balloon 54 and distal to guidewire entry port 50. One preferred balloon catheter of the present invention comprises three lumens disposed within an outer shaft 60. The three lumens include guidewire lumen 64 (defined by inner member 62) for railing the catheter over a guidewire; inflation lumen 66 for inflation of the balloon; and pressure lumen 68 for pressure measurement. Inflation lumen 66 begins at inflation syringe connector 40 and exits into balloon 54. Pressure lumen 68 begins at the pressure transducer 22 and exits in balloon 54. The inner member 62 is coaxial with one of the inflation lumen 66 or pressure lumen 68 and defines the guidewire lumen 64. In the specific embodiment depicted in FIG. 6, the inner member 62 defining the guidewire lumen 64 is coaxial with the pressure lumen 68. Guidewire lumen 64 begins at guidewire entry port 50, extends through balloon 54, and exits guidewire exit port 55 at the distal tip of the catheter. The short guidewire lumen allows the catheter to be exchanged or removed from the patient without resorting to removal of both the catheter and the guidewire as a unit or resorting to an extra length guidewire, which can be cumbersome. One suitable length for guidewire lumen 64 is about 30 cm.

The guidewire lumen 64, inflation lumen 66, and pressure lumen 68 can take any configuration and are not limited to a coaxial configuration. For example, in other embodiments, guidewire lumen 64, inflation lumen 66, and pressure lumen 68 disposed separately in a non-coaxial configuration. In yet further alternative embodiments, a single lumen catheter may be used in which fluid is infused and resulting balloon pressure is measured through the same lumen opening. In still other variations, the catheter shaft 60 includes an additional lumen that is an inflation lumen for an additional, high-pressure balloon, which could reside distally to the measurement balloon.

As indicated above and shown schematically in FIG. 1, catheter 24 typically includes a memory 30 that contains address spaces allocated for information specific to balloon catheter 24. This information can include, for example, parameters for how controller 12 should operate the inflation and deflation of the attached balloon catheter 24. This information is read from memory 30 when connected to controller 12. In one specific embodiment comprising the balloon catheter as depicted in FIG. 3, memory 30 is contained within pressure transducer connector 36. In these embodiments, as depicted in FIG. 2, memory 30 and pressure transducer 22 both interface with controller 12 through interface 23.

Information that can be included on memory 30 include, but is not limited to, the following:
the length of the balloon;
the diameter of the balloon (e.g., the outside maximum diameter of the balloon at one or more pressures);
linear equation(s) to transform the measured volume to a corresponding cross-sectional area (described below);
a transducer pressure at which the measurement of volume within the balloon is made;
one or more rates of fluid inflation and/or deflation to be used by syringe pump 18;
one or more pressures to measure cross-sectional area;
two or more pressures at which differing cross-sectional area measurements are made and a corresponding lumen compliance or elasticity is calculated;
date of manufacture; and/or
serial number.

Alternatively or in addition to the above information, memory 30 can include information that facilitates safety of the balloon catheter such as, for example, the following:
date of expiry (which can be compared to a date on memory 14 and is useful for informing the user of a potential expired catheter);
maximum allowable balloon pressure (after which the balloon will be deflated);
maximum allowable volume (useful for indicating, e.g., a leak or excessive compressible air in the catheter); and/or
maximum allowable volume infused in the absence of a corresponding change in pressure (useful, e.g., for identifying if the catheter is kinked or if something is prohibiting monitoring of a change in pressure by controller 12).

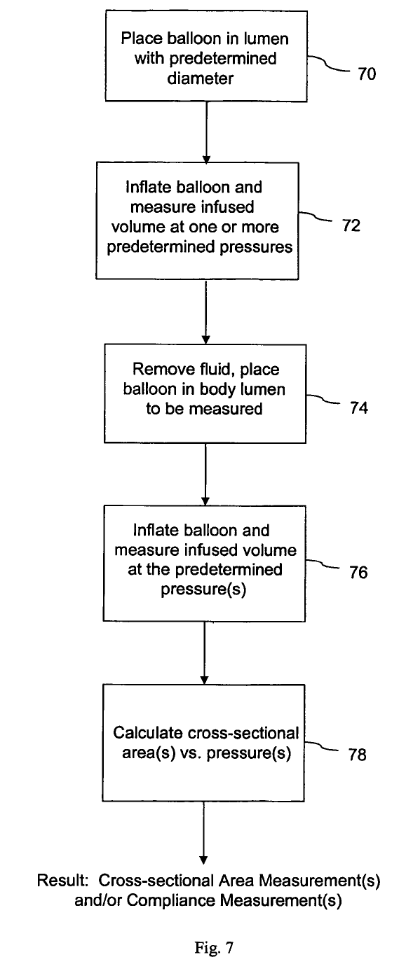

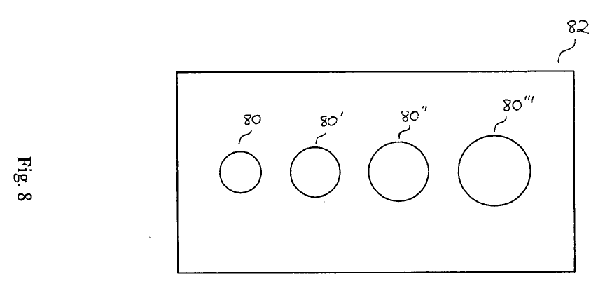

Referring generally to FIG. 7, in order to determine a physiologic characteristic of a body lumen, balloon 54 is placed in a lumen having a predetermined, fixed diameter (step 70). The balloon catheter is then inflated and infused fluid volume measurements are made successively for one or more selected balloon fluid pressures, typically in the range of 0-300 millimeters of mercury (mm Hg) (this step is also referred to herein as the “calibration cycle”) (step 72). The balloon is then deflated and inserted into the vessel or other body lumen of interest (step 74), and measurements of infused fluid volume are made again for the same successive selected balloon fluid pressure(s) (this step is also referred to herein as the “measurement cycle”) (step 76). The data from these two sets of measurements are then used to calculate vessel internal dimensions, such as cross-sectional area or diameter, at each selected balloon fluid pressure (step 78).

In certain embodiments of the method, information on memory 30 is accessed by controller 12 and used to guide operation of the system for determining a physiologic characteristic. Typically, memory 30 can be accessed by controller 12 at any point during the method described herein. In one preferred embodiment, memory 30 is accessed at all points of the method to control operation of the system. Typically, most information is obtained at initiation of any of the calibration and measurement cycles.

The lumen having the predetermined, fixed diameter (used in step 70; also referred to herein as a “fixed lumen”) can be essentially anything that is substantially non-compliant and can be placed around the deflated balloon so as to define a lumen with a known diameter. For example, the fixed lumen can be a substantially non-compliant, protective sheath or similar non-compliant tube of known diameter that fits tightly over the deflated balloon at the tip of the catheter. Alternatively, the fixed lumen can be a block of substantially non-compliant material (e.g., steel, a non-compliant polymer, or the like) with a hole defining the lumen, wherein the hole is of a known diameter and of sufficient depth for insertion of the balloon portion of the catheter. The diameter of the fixed lumen may be sufficiently small so as to constrain the balloon completely such that the balloon has essentially no volume upon inflation.

In embodiments in which the fixed lumen is a protective sheath of known internal dimensions covering the balloon, the sheath is typically assembled on the balloon during manufacturing but is removed when the catheter is ready to take a measurement in a lumen of unknown size. This lumen can also be within a stent which is crimped over the balloon where the inner lumen of the crimped stent is of known size.

In other embodiments in which the fixed lumen comprises a block of non-compliant material, the block can include two or more holes of varying diameters suitable or potentially suitable for use in accordance with the present invention. One specific variation of this embodiment is shown in FIG. 8. A plurality of fixed lumens 80, 80′, 80″, and 80′″ of known diameter in block 82 allows the system user to select the most appropriate known lumen for calibration according to, e.g., the characteristics of the balloon catheter and/or the body lumen to be measured.

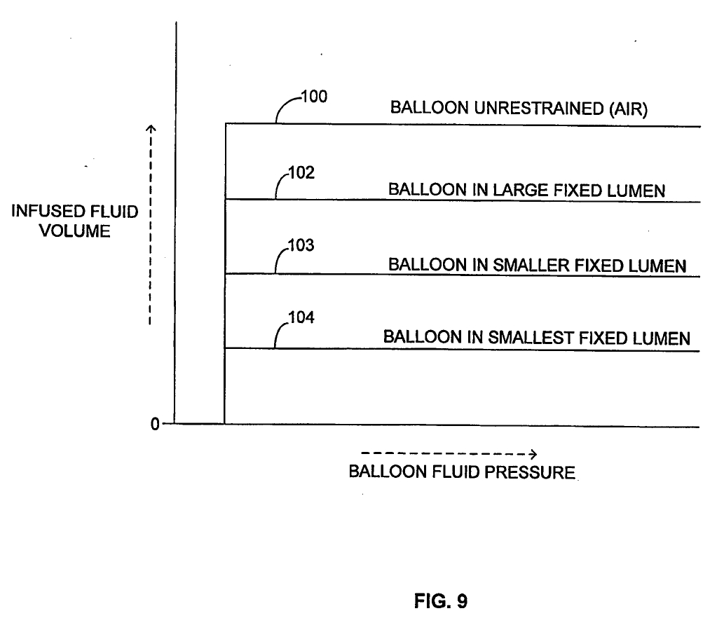

During fluid infusion of an ideal balloon catheter (made of non-compliant materials) which is unrestrained, balloon fluid pressure will typically remain near zero as fluid infusion is first initiated and then continued until the balloon is completely filled, at which point the balloon fluid pressure will increase substantially. This ideal pressure-volume response of an unrestrained balloon is shown as curve 100 in FIG. 9 and can be characterized mathematically as follows: Vₐ(P)=volume displaced by balloon−volume of balloon wall+volume of catheter.
Vₐ(P)=(πd₀²L/4)−πd₀Lt+V_{c} (Equation 1)
where Vₐ(P)=the infused fluid volume of a catheter with an unrestrained balloon at a selected pressure (P) which is greater than zero, d₀=the outside diameter of the inflated balloon at the selected pressure, L=balloon length (inflated), t=balloon wall thickness and V_{c}=the volume of the catheter lumen at the selected pressure.

FIG. 9 also shows the pressure-volume response curves for the ideal balloon catheter of curve 100 positioned in a number of different fixed lumens, all having a smaller inside diameter than the outside diameter of the fully inflated ideal balloon. In such a situation, the balloon will fill with fluid while the pressure remains near zero until the balloon contacts the internal surface of the fixed lumen, at which point the balloon fluid pressure will increase substantially. This is shown as curves 102-104 in FIG. 9.

This situation can be characterized mathematically as follows: Vₜ(P)=volume of the balloon when it contacts the inner surface of the fixed lumen−volume of balloon wall+volume of catheter lumen.
Vₜ(P)=(πdₜ²L/4)−πd₀Lt+V_{c} (Equation 2)
where Vₜ(P)=the infused fluid volume of a catheter with its balloon placed within a fixed lumen having a smaller inside diameter than the outside diameter of the fully inflated balloon at a selected pressure (P) which is greater than zero and dₜ=internal diameter of the fixed lumen.

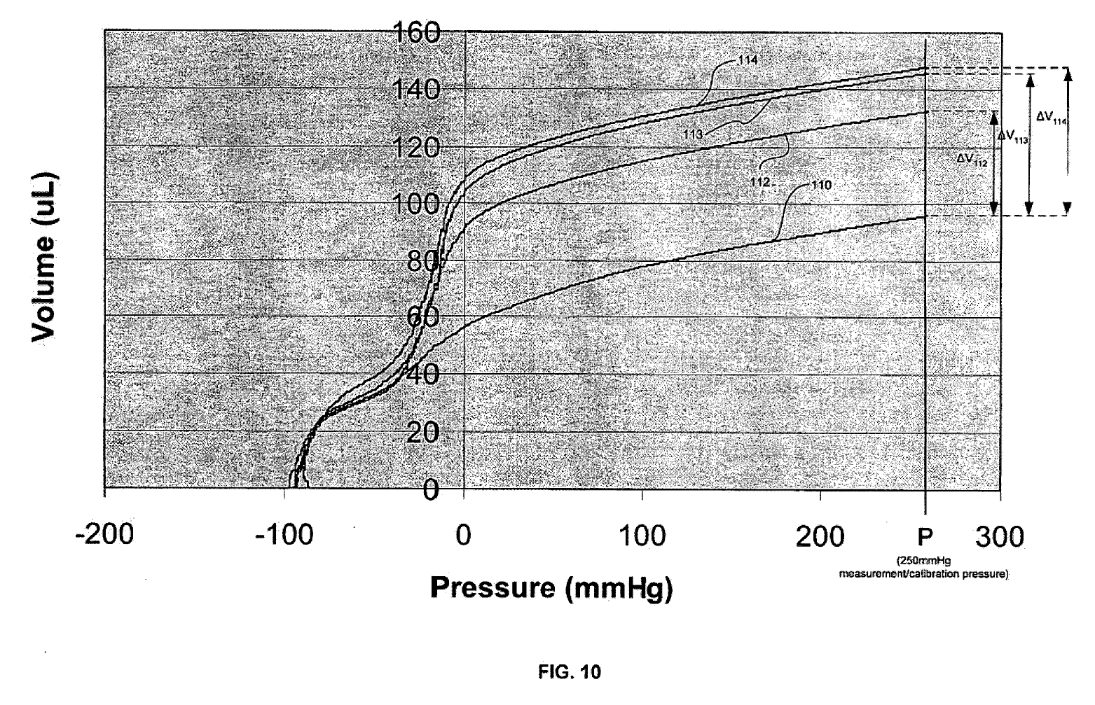

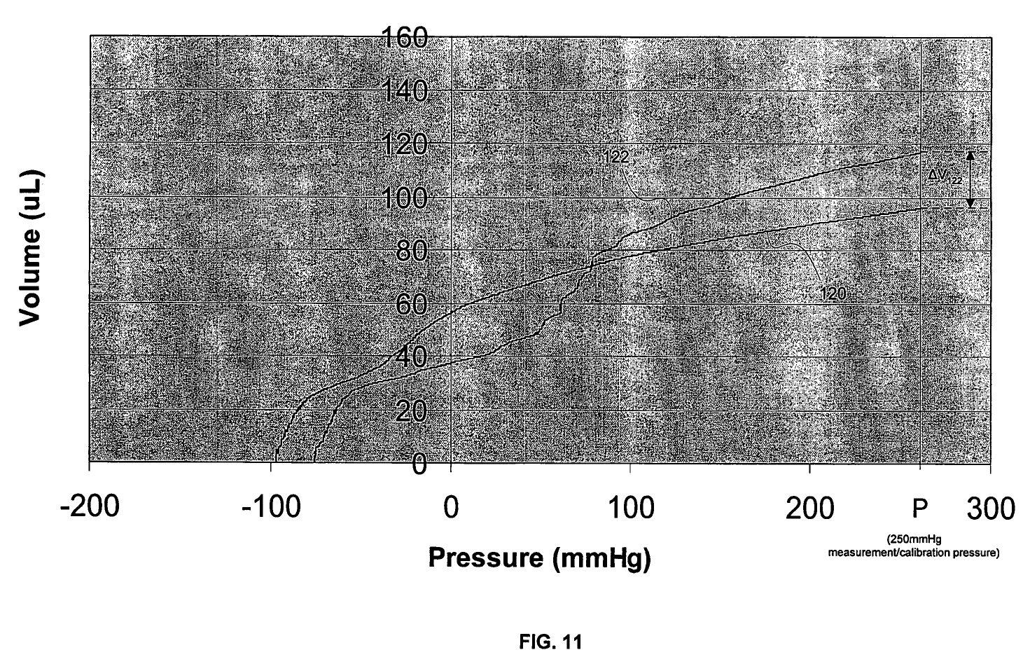

In accordance with the methods of the present invention, determination of cross-sectional area of a body lumen (e.g., blood vessel) comprises measuring the difference (ΔV) between (a) an infused fluid volume at a preselected pressure when the catheter is constrained within a fixed lumen having a known (predetermined) diameter (e.g., a rigid tube) (also referred to herein as a “calibration lumen”) and (b) an infused fluid volume at the preselected pressure when the balloon catheter is positioned at a location of interest within a body lumen having an unknown diameter:
ΔV=V_{bl}(P)−V_{cl}(P) (Equation 3)
where V_{cl}(P) is the infused fluid volume of a catheter with a balloon positioned within the calibration lumen at a selected pressure (P) which is greater than zero; and V_{bl}(P) is the infused fluid volume of the catheter with the balloon positioned at a location of interest within the body lumen at the selected pressure. This is represented graphically in FIG. 10, which shows the actual pressure-volume response curves for a balloon catheter positioned in a fixed lumen of known size (or “calibration lumen”; shown as curve 110) and a number of lumens of increasing size (shown as curves 112-114), all having a larger inside diameter than the outside diameter of the fully inflated ideal balloon. For each of curves 112-114, a ΔV with respect to curve 110 is depicted (ΔV₁₁₂, ΔV₁₁₃, and ΔV₁₁₄, respectively). FIG. 11, which shows a similar graphical representation of ΔV at measurement pressure P, depicts the actual pressure-volume response curves for a balloon catheter positioned in a fixed lumen of known size (shown as curve 120) and a body lumen (blood vessel) of a patient with a blood pressure of around 135/65 mmHg (shown as curves 122-114).

Equation 2 above can be used to characterize both the pressure-volume response of a balloon positioned within the calibration lumen as well as that of a balloon within the body lumen as follows:
V_{cl}(P)=(πd_{cl}²L/4)−πd₀Lt+V_{c} (Equation 4)
V_{bl}(P)=(πd_{cl}²L/4)−πd₀Lt+V_{c} (Equation 5)
where d_{cl} and d_{bl} are the internal diameters of the calibration lumen and the location of interest within the body lumen, respectively; and d₀, L, t, V_{c}, V_{cl}(P), and V_{bl}(P) are as previously set forth for Equations 1, 2, and 3.

Appropriate substitution of equations 4 and 5 yields the following:
V_{cl}(P)=(πd_{cl}²L/4)=V_{bl}(P)−(πd_{bl}²L/4);
hence
V_{cl}(P)−V_{bl}(P)=(πd_{cl}²L/4)−(πd_{bl}²L/4).
Since πd_{bl}²/4=the cross-sectional area at the location of interest within the body lumen (A_{bl}), the cross-sectional area at the location of interest can be calculated by dividing both sides of the equation by L as follows:
A_{bl}=πd_{cl}²/4−(V_{cl}(P)−V_{bl}(P))/L (Equation 6)
or
A_{bl}=πd_{cl}²/4−(V_{bl}(P)−V_{cl}(P))/L (Equation 7).
Because ΔV=V_{bl}(P)−V_{cl}(P), Equation 7 can also be expressed as follows:
A_{bl}=πd_{cl}²/4+(ΔV)/L (Equation 7a)
As can be seen, the cross-sectional area at a desired location of a body lumen having an unknown diameter can be determined by knowing the internal diameter d_{cl} of the calibration lumen and the length L of the balloon, and further by determining the difference in the infusion volumes V_{cl}(P) and V_{cl}(P) for the balloon catheter when positioned, respectively, in the calibration lumen and the desired location within the body lumen.

Equation 7 is most accurate when the balloon is perfectly cylindrical. Typically, the balloon of the catheter will not be perfectly cylindrical and the balloon dimension will change when it inflates and comes into contact with different lumen sizes. This problem can be addressed by parameterizing the individual characteristics of each balloon during manufacturing. In this way, individual characteristics of each balloon can be taken into account in measuring the physiological characteristic(s) of a body lumen. These parameters may be stored in memory 30, which is in communication with controller 12. When the balloon catheter is used to measure a body lumen, the parameters are retrieved from memory 30 and used to adjust the calculated cross-sectional area to determine the “actual” measured cross-sectional area.

For example, in certain embodiments, the balloon catheter is parameterized, typically during manufacture, by using the catheter to measure a plurality of known size holes (e.g., three holes) to get 2 linear equation coefficients as shown below:
Y=mX+b

The equivalent linear calibration equation:
Aₐ=mA_{c}+b (Equation 8)

where:

m is the slope.

b is the offset.

Aₐ is the actual hole area.

A_{c} is the unparameterized calculated area as calculated by using Equation 7.

To derive m and b the following equations are derived for three different holes of known sizes (Aₐ₁, Aₐ₂ and Aₐ₃)

$$\begin{matrix}{m}_{1} = \frac{{A}_{a 2} - {A}_{a 1}}{{A}_{c 2} - {A}_{c 1}} & {b}_{1} = {A}_{a 2} - {m}_{1} {A}_{c 2} \\ {m}_{2} = \frac{{A}_{a 3} - {A}_{a 2}}{{A}_{c 3} - {A}_{c 2}} & {b}_{2} = {A}_{a 2} - {m}_{2} {A}_{c 2}\end{matrix}$$

The following example shows how the actual size of a hole can be calculated using the parameterized linear coefficient value:

Parameterization:

| Actual Area (mm²) | Constrained area(mm²) |
|---|---|
| 2.00 | 2.23 |
| 3.00 | 3.45 |
| 4.00 | 4.11 |

Linear Equation:

$$\begin{matrix}{m}_{1} = \frac{3.00 - 2.00}{3.45 - 2.23} & {b}_{1} = 3.00 - 0.820 \cdot 3.45 \\ {m}_{1} = 0.820 & {b}_{1} = 0.171 \\ {m}_{2} = \frac{4.00 - 3.00}{4.11 - 3.45} & {b}_{2} = 3.00 - 1.52 \cdot 3.45 \\ {m}_{2} = 1.52 & {b}_{2} = - 2.24\end{matrix}$$

Measurement:

Using the above linear coefficients, the actual area for any measured hole can be calculated as shown in the examples below:

### EXAMPLE 1

Measured area using Equation 8=2.63 mm². Since 2.23<2.63<3.0, the first set of the linear coefficients is used to calculate the actual area as shown below:

$$Actual hole size = {m}_{1} \cdot {A}_{c} + {b}_{1}$$

$$Actual Hole size = 0.820 \cdot 2.63 {mm}^{2} + 0.171 {mm}^{2} = 2.32 {mm}^{2}$$

### EXAMPLE 2

Measured area using Equation 8=3.66 mm². Since 3.45<3.66<4.11, the second set of linear coefficients is used to calculated the actual area as shown below:

$$Actual hole size = {m}_{2} \cdot {A}_{c} + {b}_{2}$$

$$Actual Hole size = 1.52 \cdot 3.66 {mm}^{2} - 2.24 {mm}^{2} = 3.32 {mm}^{2}$$

Linear equations can be used for each of the calibration and measurement cycles to filter any noise from fluctuations of body lumen pressure (e.g., systolic and diastolic blood pressure fluctuations). Accordingly, in certain variations, the system scans and stores the pressure for every step (volume) that is infused into the catheter, and, by using linear regression analysis, the data is used to filter any noise that the body pressure effect may have on the balloon pressure. For example, a linear equation that may be used to filter body pressure fluctuations is as follows:
Linear equation: Vₙ=mPₙ+b (Equation 9)

where:
m is the slope
b is the offset
Vₙ is the set of infused volume data
Pₙ is the set of pressure reading data
Vₐᵥₑ is the mean infused volume
Pₐᵥₑ is the mean pressure reading

$$m = \frac{{\sum }_{n}^{1} ( {P}_{n} - {P}_{ave} ) \cdot ( {V}_{n} - {V}_{ave} )}{\sum {P}_{n} - {P}_{ave}}$$

$$b = \frac{{V}_{ave}}{m \cdot {P}_{ave}}$$

Using Equation 9, volume can be calculated at a given measurement pressure, which is then used to calculate, for example, the actual area and diameter of the blood vessel as set forth further herein.

Further, a coefficient of determination (R²) shows how scattered the data points are around the P/V line and can be used in both the calibration sequence and measurement sequence to determine whether the P/V line is valid:

$${R}^{2} = \frac{{\sum }_{n}^{1} {( {V}_{n} - {V}_{ave} )}^{2} - \text{(} {m}^{2} \cdot {\sum }_{n}^{1} {( {P}_{n} - {P}_{ave} )}^{2}}{( N - 2 )} \qquad ( Equation 10 )$$

If the blood pressure of the patient is too high or there is ambient noise in the system that prevents a suitable clean set of pressure data from being acquired, (e.g. other noise in the signal such as that due to motion artifact, which might prevent accurate measurements from being made), the R² value will be low and a valid measurement cannot be made. Accordingly, in certain embodiments, the system can be configured to produce an error state in which the user is alerted that the blood pressure of the patient is too high.

Actual blood vessels, of course, are typically compliant to some extent. If vessels are investigated in vivo using the system of the present invention, the cross-sectional area of the vessel would increase slightly following the infusion of additional fluid beyond that necessary to just produce contact between the balloon and the interior surface of a vessel. The change in cross-sectional area with an increase in pressure is a measure of vessel compliance or elasticity. The present invention can thus not only produce an accurate result for cross-sectional area, but also provide direct, in vivo information on vessel compliance as well.

Thus, measuring the diameter at two different inflation pressures provides a measurement of compliance. In accordance with the present invention, compliance can be expressed as the relationship between the amount of volume infused and the pressure that is needed to infuse the extra fluid in order to expand the artery. This requires expanding the artery where it is contacted by the balloon to above its nominal size, typically by a very small amount. The following equation is particularly suitable for determining compliance using the methods provided herein:

$$C = \frac{{A}_{2} - {A}_{1}}{{A}_{1} \cdot ( {P}_{2} - {P}_{1} )} \cdot 100 \%$$

where:
C is the percentage of compliance of the constrained artery for every change in pressure (P₂-P₁)
A₁ is the area measured at P₁
A₂ is the area measured at P₂
P₁ is low pressure where the balloon is touching the artery wall (e.g., 220 mmHg)
P₂ is high pressure where the balloon is slightly flex the artery wall (e.g., 260 mmHg).

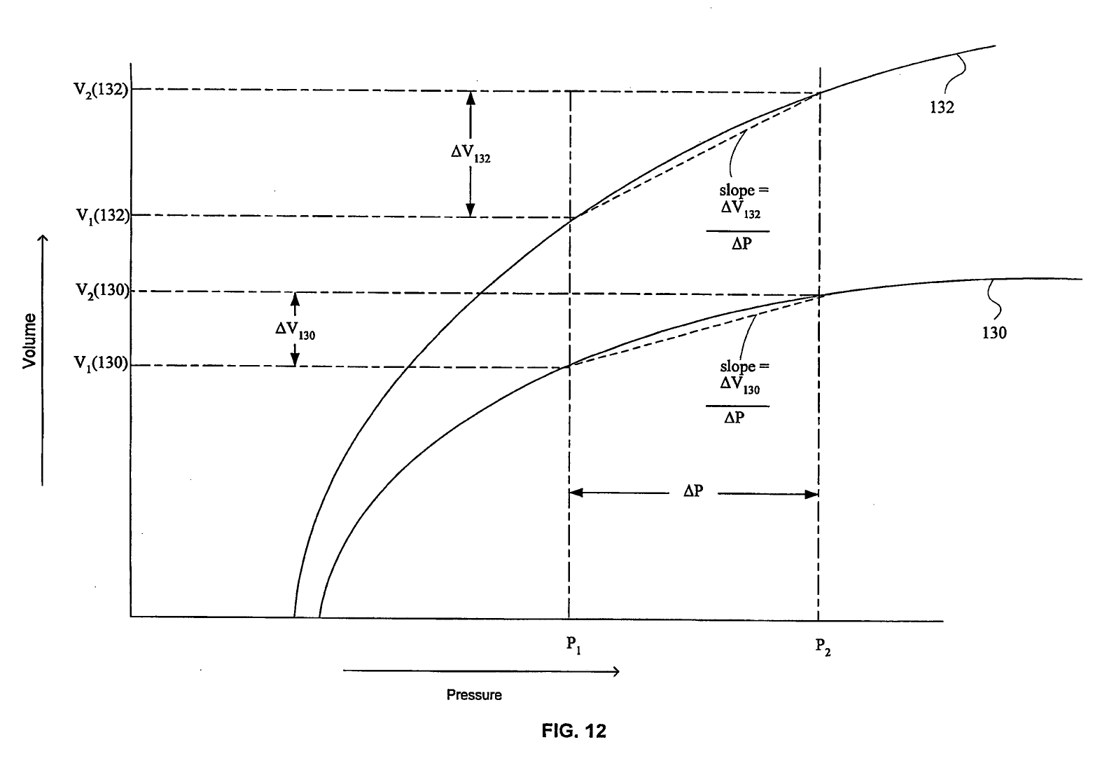

Compliance can be expressed graphically by plotting infused volume against pressure. In this case, compliance (being the overall compliance of the measurement system, which is the sum of the compliance of the catheter system, including the balloon and the inflation fluid and the compliance of the restraining lumen) is represented as the slope of the pressure/volume curve. This can be seen graphically in FIG. 12, showing the slopes of a calibration curve 130 (with the balloon in a fixed lumen of known diameter) and of a measurement curve 132 (with the balloon in a body lumen). FIG. 12 indicates points on each of curves 130 and 132 corresponding to a first volume V₁ at a first pressure P₁ and a second volume V₂ at a second pressure P₂. Compliance of catheter system while in the calibration lumen is represented as ΔV₁₃₀/ΔP, while the compliance of the catheter system while in the body lumen is represented as ΔV₁₃₂/ΔP.

In determining the compliance of a body lumen, the compliance of the calibration curve is typically taken as 0 compliance (the natural compliance of the catheter system). This compliance is a function of, for example, the materials of the catheter as well as the volume of any compressible medium (e.g., gas bubbles in the fluid) within the catheter. Accordingly, the calibration curve compliance typically depends of the specific catheter being used, on the fluid being used in the preparation (e.g., the amount of small air bubbles in the fluid), and if any small amounts of air is left in the catheter after preparation. The compliance of the body lumen (e.g., blood vessel) is calculated as the change in slope of the curve compared to the calibration slope.

The present system for determining compliance in which calibration is performed with the balloon in a fixed lumen of known diameter, provides a better means for accurately determining compliance compared to previous method in which calibration is performed with the balloon unconstrained. The use of an unconstrained balloon for calibration accounted for balloon compliance in the calculation of body lumen compliance. However, the balloon when making a measurement within a body lumen does not typically stretch or expand. Using the methods provided herein, balloon elasticity is not accounted for, thereby addressing this deficiency in previous methods.

In certain embodiments of the present method, both the actual cross-sectional area and compliance of a body lumen are measured. In one specific variation, the calibration and measurement cycles include measuring infused volume at a pressure that will be used to calculate the actual cross-sectional area of the body lumen (the “measurement pressure”); a pressure that is below the measurement pressure (the “low pressure”); and a pressure above the measurement pressure (the “high pressure,” typically just slightly above the measurement pressure). For example, in some embodiments for measuring characteristics of a blood vessel, suitable pressures include, e.g., a measurement pressure of 250 mmHg, a low pressure of 200 or 220 mmHg, and a high pressure of 260 mmHg. Measured volumes at the low and high pressures are used to calculate a change in area for determining compliance.

### EXAMPLE

One specific method of the present invention proceeds as follows. Balloon catheter 24 is purged of all air with a non-compressible fluid (e.g., saline), and balloon 54 of the catheter is placed in a lumen having a predeterminted, fixed diameter. Fluid is infused into the catheter through inflation lumen 66 as described above. Pressure transducer 22, which is in fluid communication with pressure lumen 68, produces a signal indicative of the pressure in pressure lumen 68 that represents the static pressure in the balloon. As noted above, both the fluid infusion actuator 18 and the pressure transducer 22 are in communication with controller 12, which measures infused fluid volume at one or more predetermined pressure values. A typical range of pressure values is 200-300 mmHg. The deflated balloon catheter is then positioned in a body lumen to be measured (such as, e.g., a blood vessel, the intestine, or the urethra), and the balloon 54 manipulated to the point of interest. Typically, this is accomplished by feeding the catheter over a guidewire which has been previously placed in the body for this purpose; in this case, the balloon catheter includes along its length dedicated guidewire lumen 64, separate from the fluid inflation and pressure monitoring lumens (see, e.g., FIG. 6), to facilitate this process. The infused fluid volume is then measured at the same predetermined pressure value(s). In each case, fluid is infused into the balloon in accordance with a predetermined rate schedule involving known fluid infusion rate amounts at specific pressure intervals. The vessel cross-sectional area is calculated using equation 7, either after each pressure-volume measurement or after all the pressure-volume measurements have been made. The ideal pressure for measurement will vary according to the body lumen being measured, but typically the ideal pressure is relatively low and usually does not exceed one atmosphere. In certain embodiments directed toward measurement of arterial vessels, the infused volume is determined at a pressure of approximately 250 mm Hg, which is the typical peak pressure that might be observed in the vessel during physical exertion. Determination of infused volume at a single pressure value will provide the physiologically useful cross-sectional area information concerning the vessel, while a plurality of area determinations, at two or more pressures, will provide information on the compliance of the vessel.

In certain variations of the infusion process of the present invention, infusion of fluid into the balloon catheter proceeds as follows. Starting from a negative pressure in a catheter 24 which has been purged of substantially all air by filling it with a fluid such as saline, with the plunger 74 of the syringe 20 in the withdrawn position, the infusion actuator 18 begins infusion to inflate the balloon 54 by stepping the plunger forward into the syringe. While the controller 12 monitors each step of fluid infusion, movement of the plunger may be further verified by interrogating an optical sensor at each cycle of a predetermined number of steps to ensure that a change in position has occurred. After an initial, predetermined volume of fluid has been infused, controller 12 verifies that the transducer has sensed a predetermined minimum increase in pressure. During the calibration the controller 12 monitors pressure and volume relationships and checks for leaks in or kinking of the catheter and for proper functioning of the transducer. To ensure patient safety, if controller 12 senses a problem in either the optical position sensor or the pressure sensor (for example, if proper motion of the plunger is not observed or there has not been a proper increase in pressure within the balloon), then the stepper motor is immediately reversed to withdraw the plunger and remove the infused fluid. In certain variations, the controller 12 is placed in an error state, further operation is prevented, and the user is alerted.

For example, in one specific embodiment in which infusion syringe 20 is a calibrated 3 cc syringe, infusion pump 18 begins infusion starting from a negative pressure of −100 mm Hg. Movement of the plunger is verified at each cycle of 110 steps, corresponding to 0.1375 mm of linear movement of the syringe plunger. After infusion of 40 μl of fluid, corresponding to about 550 steps, controller 12 verifies that the transducer has sensed a minimum increase in pressure of at least a 5 mmHg.

Under normal operating conditions, when the initial position and pressure indicators are correct, the system continues to step the plunger forward infusing fluid and monitoring pressure at each step until the pressure at the transducer reaches a predetermined endpoint pressure slightly greater that the pressure measurement point, or until a predetermined endpoint total of fluid has been infused (e.g., 260 mmHg, 10 mmHg greater than a 250 mmHg measurement point, or a total of 160 μl of fluid). At either of these endpoints, the stepper motor is reversed and the fluid is withdrawn until the starting position of the plunger is reached.

In certain variations, the infused volume measurement(s) are used to calculate the cross-sectional area and then the calculated cross-sectional area is transformed using one or more linear equation(s) (see, e.g., Equation 8 and related description, supra) that are specific to the individual catheter connected to the computer controller. The transformed cross-sectional area results in the true measured cross-sectional area for the specific infused amount of volume for the specific balloon catheter used for the measurement.

The area and diameter calculations taken as a whole will show increases in area and diameter with increases in pressure. A plot of this information will show the compliance of the vessel, similar to the plot of volume vs. pressure shown in FIG. 12. As indicated above, and depicted graphically in FIG. 12, compliance may be represented as the difference between the measured volumes (V₁ and V₂) at at least two measured pressures (P₁ and P₂, respectively).

Hence, an accurate and fast apparatus and method has been described which provides size and compliance information for blood vessels. Further, the apparatus can be used to obtain similar information for other body members having an opening therein (some of which are tube-like), such as the intestines, the bronchia, the urethra, the cervix, etc. This information is particularly useful in the diagnosis of certain diseases affecting vessels and such body members. The accuracy of the apparatus and method exceeds significantly that of existing methods relative to size determination. The apparatus and method further provide compliance information which heretofore has not been available.

Although a preferred embodiment of the invention has been disclosed herein for illustration, it should be understood that various changes, modifications, and substitutions may be incorporated in such embodiment without departing from the spirit of the invention which is defined by the claims which follow. All publications, patents, and patent applications cited herein are hereby incorporated by reference in their entirety for all purposes.

What is claimed is:

1. A method for determining a physiological characteristic at a location within a body lumen, said method comprising:
introducing an incompressible medium into a balloon to inflate the balloon to at least one preselected pressure while the balloon is within a calibration lumen having a predetermined, fixed diameter;
determining the internal volume of the balloon while inflated within the calibration lumen at the preselected inflation pressure;
introducing the balloon while uninflated to the location within the body lumen;
introducing the incompressible medium into the balloon to inflate the balloon to the preselected pressure at the location;
measuring the internal volume of the balloon while inflated at the location; and
determining the physiological characteristic at the location based on the difference between the volume of the balloon while inflated within the calibration lumen and the measured volume at the location.

2. The method of claim 1 wherein the internal volume of the balloon while inflated within the calibration lumen is substantially zero.

3. The method of claim 2, wherein the calibration lumen is a protective sheath or crimped stent.

4. The method of claim 3, wherein the calibration lumen has been assembled on the balloon during manufacturing of the balloon.

5. The method of claim 1 wherein the physiologic characteristic is an internal dimension.

6. The method of claim 5, wherein the internal dimension includes a cross-sectional area.

7. The method of claim 5, wherein the internal dimension includes an internal diameter.

8. The method of claim 1, wherein the physiologic characteristic is lumen compliance and the lumen compliance is determined based on a difference between a first measured volume of inflation medium at a first preselected pressure and a second measured volume at a second preselected pressure.

9. The method of claim 8, wherein the first and second preselected pressures are below 300 mm Hg.

10. The method of claim 8, which comprises calculating cross-sectional area and/or internal diameter at the first and second preselected pressures.

11. The method of claim 8, wherein the second preselected pressure is greater than the first preselected pressure, and wherein determining lumen compliance comprises calculating a percentage increase between the first measured volume and the second measured volume.

12. The method of claim 1, wherein the step of introducing incompressible medium comprises introducing the incompressible medium by positive displacement and the volume measuring step comprises measuring the volume based on the amount of incompressible medium introduced.

13. The method of claim 1, wherein the body lumen is selected from the group consisting of a blood vessel, the gastro-intestinal tract, the bronchia, the urethra, and the cervix.

14. The method of claim 1, wherein the step of introducing incompressible medium comprises introducing the medium at a substantially constant rate in the range from 4 to 100 μl/sec.

15. The method of claim 1, wherein the step of introducing incompressible medium comprises introducing the medium at a variable rate in the range from 4 to 100 μl/sec.

16. The method of claim 1, wherein the preselected pressure is below 300 mm Hg.

17. The method of claim 1, wherein the body lumen is a coronary blood vessel and the preselected pressure is between 200 and 300 mmHg.

18. The method of claim 1, wherein the body lumen is a non-vascular body lumen and the preselected pressure is below one atmosphere.

19. The method of claim 1, which comprises determining medium volume for a plurality of medium pressures below 300 mmHg.

20. The method of claim 1, further comprising the steps of stopping and then reversing the introduction of incompressible medium into the balloon at preselected values of medium pressure and volume.

21. The method of claim 20, wherein the preselected medium pressure is in the range from 0 to 400 mmHg.

22. The method of claim 21, wherein the incompressible medium is introduced by positive displacement and the volume is measured based on the amount displaced.

23. The method of claim 5, wherein the body lumen is selected from the group consisting of a blood vessel, an intestine, a bronchial tube, a urethra, and the cervix.

24. A system for measuring a physiological characteristic of a body lumen, said system comprising:
a catheter body having a proximal end, a distal end, and an inflation lumen extending from the proximal end to near the distal end;
an inflatable balloon mounted near the distal end of the catheter body and being connected to the inflation lumen;
means for introducing a volume of an incompressible medium through a proximal end of the inflation lumen to inflate the inflatable balloon;
means for measuring the pressure of incompressible medium within the balloon; and
means for determining the physiological characteristic of a location within the body lumen based on the difference between (a) the volume of inflation medium required to produce a preselected pressure of inflation medium within the balloon when the balloon is constrained within a lumen having a predetermined, fixed diameter and (b) the volume of inflation medium required to produce the preselected pressure of inflation medium within the balloon when the balloon is at the location within the body lumen.

25. The system of claim 24, wherein the means for measuring the pressure of incompressible medium within the balloon comprises means for measuring a pressure of up to about 300 mm Hg.

26. The system of claim 24, wherein the inflatable balloon when inflated is generally cylindrical and has a length in the range from about 3 mm to about 40 mm and a diameter in the range from about 1 mm to about 20 mm.

27. The system of claim 26, wherein the inflatable balloon when inflated has a length in the range from about 3 mm to about 15 mm and a diameter in the range from about 2 mm to about 10 mm.

28. The system of claim 24, wherein at least one parameter of the balloon is predetermined.

29. The system of claim 28, wherein the predetermined parameter is stored in a memory integral to the catheter body.

30. The system of claim 24, wherein the means for introducing a measured volume of inflation medium comprises a syringe.

31. The system of claim 30, wherein the syringe is coupled to a linear actuator.

32. The system of claim 24, wherein the catheter body includes at least a second lumen which is connected at its distal end to the interior of the inflatable balloon and wherein the means for measuring pressure is connected to said second lumen.

33. The system of claim 32, wherein the second lumen extends to the proximal end of the catheter, and wherein the pressure measuring means comprises a pressure sensor disposed near the proximal end of the second lumen.

34. The system of claim 24, wherein the means for measuring pressure comprises a pressure sensor disposed within the interior of the inflatable balloon.

35. The system of claim 24, wherein the means for measuring pressure comprises a pressure sensor disposed at or near the proximal end of the inflation lumen.

36. A system for connection to a balloon catheter, said balloon catheter having a proximal end, a distal end, an inflation lumen extending from the proximal end to near the distal end, and an inflatable balloon mounted near the distal end of the catheter body and connected to the inflation lumen, said system for determining a physiologic characteristic of a body lumen and comprising:
means for introducing a measured volume of incompressible medium to the balloon catheter;
means for receiving a signal corresponding to static pressure within the balloon from the catheter; and
means for calculating the physiologic characteristic based on (a) the value of a first measured volume of inflation medium at a preselected pressure when the inflatable balloon is constrained within a lumen having a predetermined, fixed diameter, and (b) the value of a second measured volume of inflation medium at the preselected pressure when the inflatable balloon is disposed within the body lumen.

37. The system of claim 36, wherein the means for introducing comprises a syringe operatively connected to a position controlled motor.

38. The system of claim 36, wherein the means for calculating comprises a microprocessor which is connected to both the positioned controlled motor and the pressure means for receiving.
